(19) United States
(12) Patent Application Publication
(10) Pub. No.: US 2011/0060037 A1
(43) Pub. Date: Mar. 10, 2011

(54) USE OF NPY Y5 RECEPTOR ANTAGONISTS FOR THE PREVENTION OF PSYCHO-STIMULANT AND OPIOID ABUSE

(76) Inventors: David Paul Drucker Woldbye, Copenhagen O (DK); Gunnar Sørensen, Copenhagen SV (DK)

(21) Appl. No.: 12/921,843

(22) PCT Filed: Mar. 11, 2009
(86) PCT No.: PCT/DK2009/050052

§ 371 Date: Nov. 18, 2010

(30) Foreign Application Priority Data
Mar. 12, 2008 (DK) PA 2008 00380

(51) Int. Cl. A61K 31/352; A61P 25/30; A61P 25/36

(52) U.S. Cl. 514/454

(57) ABSTRACT

The present invention relates to the use of NPY receptor antagonists for the preparation of a medicament for achieving a reduction in and/or prevention of abuse of psycho-stimulants, opioids and related substances.

All patent and non-patent references cited in the application, or in the present application, are also hereby incorporated by reference in their entirety.

### FIELD OF INVENTION

The present invention relates to the use of NPY Y5 receptor antagonists for reducing and/or preventing abuse of psychostimulants, opioids and related substances.

### BACKGROUND OF INVENTION

Abuse of and dependence on psychostimulants (e.g., cocaine, amphetamines), opioids (e.g., heroin, morphine), and related substances is a serious problem for most modern societies with dire consequences both at the personal and economical level. Many families and human lives have been devastated by addiction to these drugs. At present, there are no proven effective pharmacological interventions available for dependence on cocaine and other psychostimulants. The same applies to addiction to opioids. Treatment relies on existing cognitive behaviour therapies combined with contingency management strategies. Thus there is a great need for the development of novel drugs to treat addiction to these substances.

According to the ICD-10 Classification of Mental and Behavioural Disorders (WHO, Geneva, 1993) drug dependence is characterized by “Impaired capacity to control substance-taking behaviour in terms of onset, termination or level of use”. There is a “strong desire or sense of compulsion to take the substance” (i.e., craving). Substance use continues in spite of harmful consequences. The term addiction is often used interchangeably with drug dependence, but some people stress that dependence can sometimes result from legal, long-term use of medication while addiction is often characterized by a psychological need for a drug.

In the establishment of dependence to drugs of abuse, including psychostimulants and opioids, the individual goes through four specific phases: (1) the initiation phase where the person learns to associate acute positive rewarding effects with drug intake (euphoria). (2) The maintenance phase where drug intake becomes compulsive. (3) The withdrawal phase that signifies that dependence has developed. (4) The craving phase where the dependent organism will take great pains to acquire the drug for repeating intake. Craving is particularly troublesome in treatment of drug dependence because it often leads to relapse or reinstatement of drug intake even after long periods of abstinence. In fact, cravings for drugs in otherwise rehabilitated addicts are so strong, that arresting ongoing drug abuse is not considered nearly as difficult as that of preventing relapse.

Treatment of drug abuse has traditionally been focused on treating the symptoms associated with drug abuse, not on treating the disease itself. Withdrawal or abstinence syndrome is the collective term used for the symptoms associated with cessation of drug use and/or abuse. Typically the symptoms hereof are the opposite of the effect of the abused drug, for example: dysphoria, depression, and anxiety among others. Treatment of withdrawal syndrome has been directed towards relieving these symptoms with anti-depressants. Treatment of the desire to take the drugs, e.g. treating the craving has traditionally at the best been considered difficult, as it is believed that the craving is the result of a psychological addiction or dependence.

Treatment of the disease of craving for drugs, these being psychostimulants, opioids, or others, would solve the problem of relapsing into drug abuse and thus at least reduce and at best prevent abuse of these compounds.

### SUMMARY OF INVENTION

The present invention provides a means for treating the disease as well as the symptoms associated with drug use and especially drug abuse.

The present study demonstrates the surprising finding that administration of NPY Y5 receptor antagonists causes a significant reduction in acute self-administration of psychostimulants. This shows that NPY Y5 receptor antagonists block the acute rewarding effects of the psychostimulants and is consistent with the concept that Y5 antagonists are effective for treating addiction as characterized by the craving for drugs.

Furthermore, the maximal achievable effect of the psychostimulants tested appeared to be decreased by the administration of NPY Y5 antagonists. This is surprising because it suggests that with the therapeutic use of Y5 antagonists for treatment of psychostimulant, opioid and abuse of other drugs, the patients are not likely to increase their ingestion of cocaine by taking higher, potentially lethal doses.

The present study also shows that treatment with the Y5 antagonist significantly reduces relapse to cocaine-induced conditioned place preference (CPP) and is associated with faster extinction of the CPP response. Conditioned place preference (CPP) is one of the most popular models to study the motivational effects of drugs and non-drug treatments. The fact that cocaine relapse was antagonized in the CPP model shows that treatment with NPY Y5 antagonists are particularly useful in preventing relapse to cocaine seeking behavior. As stated above, preventing relapse is the key problem in treating psychostimulant and opioid addiction, as opposed to merely treating the symptoms associated with drawal. Faster extinction associated with NPY Y5 antagonist treatment in the CPP model furthermore shows that NPY Y5 antagonists are useful for stopping ongoing abuse of psychostimulants and/or opioids.

Thus the present invention discloses the use of a NPY Y5 receptor antagonist for the preparation of a medicament for achieving a reduction in and/or prevention of abuse of psychostimulants, opioids and/or other drugs/compounds in an individual.

It is also an aspect of the present invention to provide a medicament comprising a compound capable of reducing and/or preventing abuse of psychostimulants, opioids and/or other drugs/compounds in an individual.

A kit of parts comprising the medicament as herein disclosed is yet an aspect of the present invention.

### DESCRIPTION OF DRAWINGS

FIG. 1 Dose-dependent reduction of acute cocaine self-administration.

FIG. 2 Effect of L-152,804 on the self-administration dose-response curve.

FIG. 3 Effect of L-152,804 pre-treatment.

FIG. 4 Self-administration of cocaine in WT and Y5-KO mice.

FIG. 5 Effects of Y5 antagonist on cocaine induced conditioned place preference (CPP).

FIG. 6 Cocaine-induced conditioned place preference studied in Y5-KO and WT mice

FIG. 7 Cocaine induced significant hyperactivity in vehicle pre-treated mice but not in mice pre-treated with the Y5 antagonist, L-152,804.

FIG. 8 Cocaine-induced hyperactivity was significantly attenuated in Y5-KO as compared to WT mice.

### DEFINITIONS

The following definitions are provided to simplify discussion of the invention. They should not, therefore, be construed as limiting the invention, which is defined in scope by the appended claims and the following description.
Agonist: A compound/ligand capable of binding a receptor eliciting a biological response.
Antagonist: A compound capable of binding a receptor having the effect of blocking a biological response. Here especially a compound that is a ligand/binds a NPY Y5 receptor in a manner that does not activate a biological response itself upon binding to a receptor, but blocks or dampens agonist-mediated responses.
Drug: Used interchangeably with compound, substance, medicine, remedy, ingredient or preparation. Here the term generally is used to cover psychoactive/psychostimulatory compounds and opioids.
Drug abuse: Non-medical or recreational use of especially psychoactive compounds.
Drug use: Medical use of a drug/compound, generally a use in accordance with guidelines set forth by a medical practitioner.
Individual: A single member of a species, herein preferably a mammalian species.
Medicament: A pharmaceutical formulation comprising at least one active ingredient.
Opioid: Opioids are compounds that bind the opioid receptors
Prevention of abuse: The cessation of abuse of a drug.
Psychoactive substance: A substance that acts primarily upon the central nervous system where it alters brain function, resulting in temporary changes in perception, mood, consciousness and behaviour. The terms psychoactive and psychostimulatory are interchangeable.
Psychostimulant: A drug that temporarily increases alertness, awareness, give rise to euphoria and/or other sensations arising from an increased activity of the sympathetic nervous system, the central nervous system or both.
Reduction of abuse: Abuse of a drug to a lesser extent than prior to administration of the medicament of the invention. The lesser extent is generally a lower dosage of a drug abused, but may also reflect the behavioral pattern of fewer intakes of a drug.
Relapse: A return to drug use/abuse after a cessation attempt.
Second active ingredient: A pharmaceutically active compound other than the primary active compound which herein is a NPY Y5 receptor antagonist.

### DETAILED DESCRIPTION OF THE INVENTION

### Neurobiology of Addiction to Psychostimulants, Opioids, and Related Substances

Many regions in the brain are involved in mediating the development of drug dependence and addiction. It appears that different brain regions are involved in mediating the different phases of dependence described above. However, one key region that seems to be particularly important in mediating the reinforcing effects of drugs is the nucleus accumbens (NAc) located in the ventral striatum of the brain. The NPY Y5 receptor of relevance to this invention is found in the (NAc). NAc is also part of the so-called extended amygdala, a group of brain structures that all appears to play an important role in drug addiction. This NAc receives neuronal input from mesolimbic dopamine neurons in the ventral tegmental area. Increased extracellular levels of the neurotransmitter dopamine in the NAc have repeatedly been associated with intake of drugs of abuse. The NAc can be divided into two parts: the shell and the core. Particularly, dopamine in the shell region has been implicated in drug reward processes. Dopamine in the NAc appears to mediate salience of events experienced by an organism (drug intake), causing events to become attention grapping.

Both psychostimulants and opioids are believed to cause acute rewarding effects by increasing extracellular levels of dopamine in the NAc. Psychostimulants cause this rewarding effect either by inhibiting the reuptake of dopamine or by stimulating the release of dopamine from dopamine-secreting neurons. Opioids act by binding to endogenous opioid G-protein coupled receptors (mu, delta, kappa) located on neurons in many brain regions, including the ventral tegmentum dopamine neurons releasing dopamine in the NAc. The mu-receptors appear to be particularly important for acute rewarding effects.

### The NPY Y5 Receptor and Neuropeptide Y (NPY)

The NPY Y5 receptor is one of at least five G-protein coupled receptors (Y1, Y2, Y4, Y5, Y6), to which the neurotransmitter or neuromodulator NPY binds. NPY is the major endogenous ligand for the NPY Y5 receptor. NPY is widely distributed in the human central nervous system, including the NAc. NPY is member of a polypeptide family also including peptide YY and pancreatic polypeptide. NPY and other members of the polypeptide family act by binding to the G-protein coupled receptors of above causing a reduction in cyclic AMP. NPY receptor stimulation also seems to affect G-protein inward rectifying potassium channels and intracellular calcium levels. In the brain, Y1, Y2, and Y5 are the predominant NPY receptors.

Since the Y5 receptor was cloned (Gerald et al., Nature 1996, 382:168-71), a number of selective ligands have been developed with affinity for the Y5 receptor. Agonists at Y5 receptors are peptide ligands (e.g., [hPP1-17, Ala31, Aib32]hNPY, [cPP1-7, NPY19-23, Ala31, Aib32, Gln34]hPP) that elicit an intracellular response after binding to the receptor (e.g., reduction in cyclic AMP). Several selective non-peptide Y5 antagonists have been developed to date (see below). Unlike Y5 agonists, Y5 antagonists do not elicit an intracellular response, but block binding of the endogenous ligand NPY to the receptor. A recent study indicated that the Y5 receptor may form heterodimers with Y1 receptors, potentially affecting the pharmacology of Y5 antagonists in a profound manner (Gehlert et al., Biochem. Pharm. 2007, 74:1652-64).

The NAc contains NPY-secreting neurons as well as Y5, Y1, and Y2 receptors (Wolak et al., J. Comp. Neurol. 2003, 464:285-311). This suggests that NPY could affect dopamine release in the NAc, potentially mediating rewarding effects. Indeed, NPY administration directly into the NAc has been shown to cause place-preference in rats (Josselyn and Beninger, 1993). It is generally believed that the ability of a drug to cause place-preference in animals indicates that the drug possesses rewarding properties. The effect of NPY on place-preference was blocked by pretreatment with the dopamine receptor blocker cis-flupenthixol, indicating that the rewarding effects of NPY in the NAc are mediated through dopamine. At present, the NPY receptors mediating the rewarding effects of NPY in the place-preference model remain to be explored.

### Biological Effects of NPY

NPY has been implicated in numerous biological effects, including feeding, anxiety, epilepsy, regulation of blood pressure and depression. Some studies also suggest a role for NPY in mediating effects of psychostimulants. For instance, repeated administration of cocaine and other psychostimulants was shown to cause reductions in NPY levels in the frontal cortex, NAc, and/or dorsal striatum (Wahlestedt et al., PNAS 1991, 88:2078-82; Westwood & Hanson. JPET 1999, 288:1160-6). NPY has anxiolytic-like and anti-depressant-like effects, and it has been suggested that the psychostimulant-induced decrease in NPY is involved in mediating anxiety- and depression-like symptoms during withdrawal from a cocaine binge.

There is sparse data on effects of opioids on the NPY/NPY Y5 receptor system. However, one study showed that NPY agonists administered into the central nervous system might attenuate opioid withdrawal symptoms via receptors with an Y5-like receptor profile (Woldbye et al., JPET 1998). Confirming the role of Y5 receptors in attenuating withdrawal awaits the testing of specific Y5 antagonists since the former study failed to conclusively show involvement of Y5 receptors. Moreover, since different brain regions are involved in mediating opioid withdrawal symptoms (e.g., the locus coeruleus) as compared to rewarding effects of opioids (e.g., NAc), it is nowhere clear that Y5 antagonists should be effective at reducing and/or preventing abuse of opioids from the literature. In fact, from a theoretical perspective Y5 antagonists are predicted to worsen opioid withdrawal symptoms and make it more difficult for a person to stop ongoing opioid addiction.

Therefore the findings cited in the Example section of the present disclosure are highly surprising as they report that treatment with NPY Y5 receptor antagonists:
causes a significant reduction in acute self-administration of psychostimulants.
significantly reduces relapse to cocaine-induced conditioned place preference (CPP) and is associated with faster extinction of the CPP response.

### Compounds for Use as NPY Y5 Receptor Antagonists

The NPY Y5 receptor may bind compounds that are agonists, inverse agonists, or antagonists. Any compound that antagonizes the NPY Y5 receptor is of interest to the present invention. By antagonist is herein understood a compound that is a ligand/binds to the NPY Y5 receptor in a manner that does not activate a biological response itself upon binding to a receptor, but blocks or dampens agonist-mediated responses. Antagonists have affinity but no efficacy for their cognate receptors and binding will disrupt the interaction and inhibit the function of an agonist or inverse agonist at the receptors. Antagonists mediate their effects by binding to the active site or to allosteric sites on receptors or they may interact at unique binding sites not normally involved in the biological regulation of the activity of the receptors. Antagonist activity may be reversible or irreversible depending on the longevity of the antagonist-receptor complex which in turn depends on the nature of antagonist receptor binding. The majority of drug antagonists achieve their potency by competing with endogenous ligands or substrates at structurally defined binding sites on receptors.

NPY Y5 antagonists of special interest to the present invention include, but are not limited to the compounds mentioned herein below including salts and/or derivates of these compounds (synonyms in parenthesis):
1) L-152,804 (2-(3,3-dimethyl-1-oxo-4H-1H-xanthen-9-yl)-5,5-dimethyl-cyclohexane-1,3-dione) originally produced by Banyu.
2) MK-0557 (trans-N-[1-(2-fluorophenyl)-3-pyrazolyl]-3-oxospiro[6 azaisobenzofuran-1(3H),10-cyclohexane]-40-carboxamide) originally produced by Merck.
3) The 2-methanesulfonamidephenyl)piperazine class of Y5 antagonists.
4) N-[5-(4-chlorophenyl)-1Hpyrazol-3-yl]-2-indanecarboxamide.
5) FMS586 (3-(5,6,7,8-tetrahydro-9-isopropyl-carbazol-3-yl)-1-methyl-1-(2-pyridin-4-yl-ethyl)-urea Hydrochloride).
6) 3-(5-benzoyl-thiazol-2-ylamino)-benzonitrile.
7) CGP71683A and derivatives, e.g., Novartis-1 ((trans-2-nitrobenzene-2-sulphonic acid (4-(2-naphthylmethylamino)methyl)cyclohexyl methyl)amide.
8) 5,6-dihydro-4H-3-thia-1-aza-benzo[e]azulen- and 4,5-dihydro-6-oxa-3-thia-1-aza-benzo[e]azulen derivatives.
9) Triazine derivates as described in EP1183245.
10) N-(Sulfonamido)alkyl[tetrahydro-1H-benzo[e]indol-2-yl]amines, also known as 1,3-Disubstituted-5-aminopyrazoles (Kordik et al., Biorg. Med. Chem. Lett. 2001, 11:2283-6).
11) Arylsulfonamidomethylcyclohexyl derivatives: including (a) trans-N-(4-[(Quinolin-3-yl)aminocarbonyl]cyclohexylmethyl)-2,4-dichlorobenzenesulfonamide (42) (Moreno et al., Eur. J. Med. Chem., 2004, 39:49-58), (b) trans-N-(4-[N′-(pyridine-3-carbonyl)-hydrazino-carbonyl]cyclohexylmethyl)-2,4-dichloro-benzenesulfonamide (Galiano et al., Arzneimittelforschung 2005, 55:81-5).
12) 2-[4-(8-methyl-2-oxo-4H-benzo[d][1,3]oxazin-1-yl)piperidin-1-yl]-N-(9-oxo-9H-fluoren-3-yl)acetamide 5p (Torrens et al., J. Med. Chem. 2005, 48:2080-92).
13) 5,5-diphenylimidazolones: including 3-pyridyl analog 46 (Bioorg. Med. Chem. 2006, 14:5517-26).
14) Cyclohexylureido NPY Y5 receptor antagonists developed by modifying a biaryl urea lead: including cyclohexylurea 21c (Li et al., Bioorg. Med. Chem. Lett. 2008, 18:1146-50).
15) S 25585.
16) 3-amido-9-ethylcarbazoles.
17) JCF109.
18) GW438014 A.

It is an object of the present invention to provide a NPY Y5 receptor antagonist for the preparation of a medicament for achieving a reduction in and/or prevention of abuse of psychostimulants and/or opiods in a mammal. Preferably, the NPY Y5 receptor antagonist is selected from any of the above mentioned NPY Y5 receptor antagonist, or salts or derivates thereof. More preferably, the compound is L-152,804 or MK-0557, or any salts or derivates thereof. Most preferably the compound is L-152,804.

### Psychostimulants and Opioids

Psychostimulants, opioids and related compounds that fall under the category of psychoactive substances have been used and abused for centuries for both medical and recreational purposes.

Psychoactive substances are used for their mood and perception altering effects, including those with accepted uses in medicine and psychiatry. Classes of drugs frequently used recreationally include: Stimulants, which elevate the central nervous system, these are used recreationally for their euphoric and performance-enhancing effects; Hallucinogens, which induce perceptual and cognitive distortions; Hypnotics, which are used recreationally to because they induce inebriation; Analgesics, which are used recreationally because of their euphoric effects. All psychoactive substances that elicit a positive response in the user, such as euphoria, elatedness, increased awareness, heightened perception, inebriation and so on are relevant to the present invention. Drugs/compounds that give rise to a positive effect are herein generally referred to as psychostimulants, thus there is an overlap between the terms psychostimulant and opioid.

Psychostimulants, also known as psychomotor stimulants, are drugs that temporarily increase alertness, awareness, give rise to euphoria and/or other sensations arising from an increased activity of the sympathetic nervous system, the central nervous system or both, and include compounds that range from caffeine to cocaine, see below. All psychostimulants described herein are aspects of the present invention. Psychostimulants abused by humans include cocaine, D-amphetamine, methamphetamine, 3,4-methylene-dioxy-methamphetamine, and other substances. Psychostimulants share similar pharmacological properties and have high abuse potential. Particularly cocaine is well known for its ability to cause dependence and addiction. Abusers of cocaine typically go through binges to obtain euphoria where the drug is typically administered every 10 min for up to a week. Within binges, euphoria is replaced by dysphoria, triggering more drug-taking. Withdrawal from chronic cocaine use is associated with few physical signs, but a number of psychological symptoms occur that motivate the individual to relapse into drug-taking, e.g., depression, anxiety, dysphoria, insomnia, and especially a powerful craving for the drug.

Examples of psychostimulants include but are not limited to: Phenethylamines such as dopamine, epinephrine, norepinephrine; ephedrine, pseudoephedrine, cathinone, cathine, amphetamines and substituted amphetamines (alpha-methyl-phenethyl-amines): amphetamine, methamphetamine, MDA, MDMA, MDEA, MBDB, MDMC, DOM, DOB, DOI; 3,4-MDMA (“ecstasty”), Br-DragonFLY, TMA-2, TMA-6, methyl-phenidate and 2C, mescaline, dextroamphetamine, Norepinephrine and Dopamine Reuptake Inhibitors (NDRIs); bupropion, MDPV, pyrovalerone, mazindol and pipradrol, cocaine and other tropane derivative drugs related to cocaine such as troparil and lometopane, ampakines such as modafinil, adrafinil, ampalex, CX717 and Carphedon. Any psychostimulants that may be used and/or abused is an object of the present invention. Preferably, the psychostimulant abused is cocaine, D-amphetamine, methamphetamine, or 3,4-methylene-dioxy-methamphetamine. Most preferably the psychostimulant abused is cocaine.

Opioids are compounds that bind the opioid receptors and include: natural opiates such as alkaloids contained in the resin of the opium poppy including morphine; semi-synthetic opiates; fully synthetic opioids and endogenous opioid peptides, produced naturally in the body. Any opioid herein disclosed in an object of the present invention. Opioids abused by humans include heroin, morphine, methadone, and more, see herein below. Like the psychostimulants, opioids share most pharmacological effects and have high abuse potential. Particularly heroin is well known for its high abuse and dependence potential due to its high lipid-solubility that causes it to enter the brain faster than most other opioids. Administration of opioids causes euphoria that may lead to opioid dependence upon even one single administration. By definition, dependence has developed when characteristic physiological withdrawal symptoms appear upon termination of opioid-taking. These include diarrhoea, emesis, gooseflesh, yawning, and restlessness. Psychological symptoms are also prominent, e.g., dysphoria accompanied by depressive-like and anxiety-like symptoms, and especially drug craving. The dysphoria of opioid withdrawal will often compel the addict to re-administer the drug and maintain opioid dependence. Without treatment, most withdrawal symptoms are over in approximately 10 days; however, some residual clinical signs (e.g., hyperthermia, increased blood pressure and respiratory rate) may persist for months.

Opioids of relevance to the present invention include, but are not limited to the group comprising: natural opiates: such as morphine, codeine, thebaine, papaverine, and noscapine; semi-synthetic opiates, created from the natural opioids, such as hydromorphone, hydrocodone, oxycodone, and heroin; fully synthetic opioids, such as fentanyl, pethidine, methadone, and propoxyphene; endogenous opioid peptides, produced naturally in the body, such as endorphins, enkephalins, dynorphins, and endomorphins, furthermore, buprenorphine, methadone, sufentanil, remifentanil, ketobemidone, nicomorphine, pethidine, tramadol, dextropropoxyphene, pentazocine, cyclazocine, etorphine, and related substances. Preferably, the opioid abused is heroin, morphine, methadone, fentanyl, sufentanil, remifentanil buprenorphine, codeine, ketobemidone, hydromorphone, nicomorphine, oxycodone, pethidine, tramadol, dextropropoxyphene, pentazocine, cyclazocine, or etorphine. Most preferably, the opioid abused is heroin or morphine.

Abuse of any of the above compounds or related substances is reduced and/or prevented by the administration of the medicament of the present invention which comprises a NPY Y5 receptor antagonist.

Compounds or substances related to any of the herein cited psychostimulatory compounds or opioids are also of relevance to the present invention. These include but are not limited to: arecoline, cannabinoids, barbiturates, hallucinogens, tryptamines, LSD, phencyclidine (PCP), psilocybin, Serotonergic psychedelics (serotonin 5-HT2A receptor agonists), Indoles (these may also be considered to be tryptamines), Tryptamine, Dimethyltryptamine (DMT), 5-MeO-DMT, Bufotenine, Alphamethyltryptamine (AMT), 5-MeO-AMT, Dipropyltryptamine (DPT), DIPT, 5-MeO-DIPT, Psilocin, 4-HO-DIPT, Lysergamides, LSD (acid), Ergine (LSA), Ergonovine, Ibogoids, Ibogaine, Voacangine, Beta-carbolines, Harmaline, Harmine, Salvinorin A, Phenethylamines, Substituted phenethylamines, Mescaline, 2C-B, 2C-B-FLY, 2C-C, 2C-1,2C-E, 2C-T-2, 2C-T-4, 2CT-7, 2C-T-21, 2C-D, 2C-N, 2C-T-8, 3C-E, 4-FMP, Cannabinoids (CB-1 cannabinoid receptor agonists), THC, cannabis, marijuana, Myristicin, Elemicin, Cryogenine (Vertine), Bupropion, Cathine, Cathinone, Clenbuterol, DESOXY, Diethylcathinone, Dimethylcathinone, Dopamine, Br-DFLY, Ephedrine, Epinephrine, Escaline, Fenfluramine, Levalbuterol, Levmetamfetamine, MDPV, Methcathinone, Methylphenidate, Norepinephrine, Phentermine, Salbutamol, Tyramine, Venlafaxine, catecholamines, ephedrine, pseudoephedrine, and methylphenidate.

It is an object of the present invention to provide a NPY Y5 receptor antagonist for the preparation of a medicament for achieving a reduction in and/or prevention of abuse of any of the herein disclosed psychostimulants and/or opioids.

Any of the above-mentioned compounds/drugs may be used/abused in combination with each other or other drugs/stimulants. Non-limiting examples of poly drug use include: tobacco and marijuana; marijuana and coffee; LSD and ecstasy; Psychedelic mushrooms and ecstasy; cocaine and heroin or morphine (known as a “speedball”); ecstasy and viagra; ecstasy and ketamine; ecstasy and phencyclidine; ecstasy and mescaline; and heroin and diphenhydramine. Poly drug use may result in a codependency of several different compounds including tobacco and nicotine, and may thus warrant complex treatment. It is an object of the present invention to provide a medicament comprising a NPY Y5 receptor antagonist for the treatment of drug abuse characterized by craving. The medicament may be as disclosed in the below apart from at least one NPY Y5 receptor antagonist further comprise a second active ingredient that is capable of reducing/alleviating/treating a drug abuse characterized in being a poly drug use.

### Individual

The individuals herein referred to are single members of a species, preferably a mammalian species. Any mammalian species is an object of the present invention, although any of the following species are of particular relevance: mouse, rat, guinea pig, hamster, rabbit, cat, dog, pig, cow, horse, sheep, monkey, and human. Most preferably the individual of the present invention is a human. The individuals may in the present text also be referred to as patients. The term human as used herein covers any pre- or post-natal human being, thus including neonates, and wherein the human being is of any age or sex.

The individual may have a history of abusing drugs such as psychostimulants and/or opioids as herein described. A history of drug abuse may be an abuse sustained over a short or prolonged time period and relates to both a small, moderate and substantial abuse. Individuals with a history of drug abuse may have received medical or other attention due to the abuse, and may be considered detoxified and/or in rehabilitation or considered rehabilitated. Such persons are in constant danger of slipping or relapsing. A slip may be considered as an individual who once or a few times takes and/or abuses one or more psychostimulants and/or opioids. A relapse implies a return to previous behaviour patterns with repeated abuse of psychoactive substances such as psychostimulants and/or opioids.

An individual who for medical reasons has been given psychostimulants and/or opiods for analgesic or other purposes may also be a person in need of treatment according to the present invention. Several of the psychoactive compounds disclosed in the above are used for medical purposes such as to increase alertness, concentration and physical endurance. They may be prescribed to counter the effects of narcolepsy, to help patients with learning disabilities such as ADD and ADHD, depression, treatment of post-traumatic stress disorder, for use as analgesics and for use in palliative care.

Both individuals, who have a history of drug use/abuse, are rehabilitated and/or detoxified and individuals who have never been exposed to psychostimulants and/or opioids are individuals that may benefit from treatment with a medicament comprising a NPY Y5 receptor antagonist for achieving a reduction in and/or prevention of abuse of psychostimulants, opioids or other psychoactive substances. Preferably, the individual to be treated with the medicament of the present invention is a person in danger of a relapse. Also it is preferred that the individual is in danger of relapsing into an abuse of cocaine, heroin, amphetamine or derivates hereof. Most preferably, the individual is in danger of relapsing into an abuse of cocaine.

### Pharmaceutical Composition

### Administration Forms

The medicament of the present invention, which comprises a NPY Y5 receptor antagonist, may be administered by any method known in the art. The main routes of drug delivery in respect to the present invention are oral, rectal, intrathecal, or systemical via subcutaneous, intramuscular or intravenous routes, or topical, as will be described below. Other drug-administration methods, such as inhalation are also contemplated.

The compounds according to the invention may be administered with at least one other compound. The compounds may be administered simultaneously, either as separate formulations or combined in a unit dosage form, or administered sequentially. The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, patches, and powders in vials or ampoules.

### Formulations

While it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier therefore. Thus, it is an object of the present invention to provide means for comprising at least one NPY Y5 receptor antagonist in a form suitable for its administration as a medicament.

An aspect hereof is the use of pharmaceutically acceptable carriers. The choice of carrier depends upon the form the compound of the invention will be delivered in. Thus the carrier may be a solid carrier, a liquid carrier, they may include flavoring agents, lubricants, solubilizers, and the like. The carrier or excipient may include time delay material well known to the art.

The NPY Y5 antagonists of the present invention are either on their own, or aided by the excipients of the chosen pharmaceutical form, or aided by other means as described in the below, are capable of crossing the blood-brain barrier. Mechanisms for drug targeting in the brain involve going either “through” or “behind” the blood-brain barrier. Modalities for drug delivery through the blood-brain barrier entail its disruption by osmotic means, biochemically by the use of vasoactive substances such as bradykinin, or even by localized exposure to high intensity focused ultrasound (HIFU). Other strategies to go through the blood-brain barrier may entail the use of endogenous transport systems, including carrier-mediated transporters such as glucose and amino acid carriers; receptor-mediated transcytosis for insulin or transferrin; and blocking of active efflux transporters such as p-glycoprotein. Strategies for drug delivery behind the blood-brain barrier include intracerebral implantation and convectionenhanced distribution. Novel strategies include the use of nanoparticles and/or liposomes as a method of delivering the compounds of the invention to the brain.

### Pharmaceutically Acceptable Salts

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the NPY Y5 antagonist and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent. Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The NPY Y5 antagonists of the invention may be administered in the form of a salt, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

### Oral Administration Forms

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 100% by weight of a compound or compounds of the invention, with the remainder consisting of suitable pharmaceutical excipients. The pharmaceutical preparation may contain the compound related to the present invention in an amount of from 1.0 to 75% by weight, preferably from 1.0 to 60% by weight, with respect to the total preparation. For oral administration, excipient include pharmaceutical grades of mannitol, lactose, starch, magnesium stearate, sodium saccharine, talcum, cellulose, glucose, gelatine, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably contain from one to about seventy percent of the active compound. Suitable carriers are magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100 C. for half an hour. Alternatively, the solution may be sterilized by filtration and transferred to the container aseptically.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations.

### Parenteral Administration Forms

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Topical Administration Forms

The compounds of the invention can also be delivered topically. Regions for topical administration include the skin surface and any mucous membrane of a mammal such as the tissues of the vagina, rectum, nose, mouth, and throat. Compositions for topical administration via the skin and mucous membranes should not give rise to signs of irritation, such as swelling or redness. The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, or transdermal patch for example. Methods for preparing such compositions are well known in the pharmaceutical industry.

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like. Transdermal delivery is accomplished by exposing a source of the complex to a patient's skin for an extended period of time. Transdermal patches have the added advantage of providing controlled delivery of a pharmaceutical agent-chemical modifier complex to the body. Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

The compounds of the present invention may be formulated for administration as suppositories or for vaginal administration. Pessaries, tampons, creams, gels, pastes, foams or sprays containing in addition to the active ingredient such carriers as are known in the art to be appropriate.

### Nasal Administration

The compounds of the present invention may be formulated for nasal administration. The solutions or suspensions are applied directly to the nasal cavity by conventional means, for example with a dropper, pipette or spray, or as a powder. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

### Controlled Release Formulations

The NPY Y5 antagonists of the present invention may be comprised within pharmaceutical formulations that enable controlled release of the active compound. Such controlled release formulations include, but are not limited to: continuous release, controlled release, delayed release, depot, gradual release, long-term release, programmed release, and so forth. The various controlled release technologies cover a very broad spectrum of drug dosage forms. Controlled release technologies include, but are not limited to physical systems and chemical systems. Physical systems include, but are not limited to, reservoir systems with rate-controlling membranes, such as microencapsulation, macroencapsulation, and membrane systems; reservoir systems without rate-controlling membranes including those systems physically dissolved in non-porous, polymeric, or elastomeric matrices; and other physical methods, such as osmotic pumps, or adsorption onto ion-exchange resins. Chemical systems include, but are not limited to, chemical erosion of polymer matrices (e.g., heterogeneous, or homogeneous erosion), or biological erosion of a polymer matrix (e.g., heterogeneous, or homogeneous). Controlled release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

While a preferable mode of controlled release drug delivery will be oral, other modes of delivery of controlled release compositions according to this invention may be used. These include mucosal delivery, nasal delivery, ocular delivery, transdermal delivery, parenteral controlled release delivery, vaginal delivery, rectal delivery and intrauterine delivery. All of these dosage forms may be manufactured using conventional techniques, together with the techniques discussed herein.

It is thus an object of the present invention to use an NPY Y5 receptor antagonist for the preparation of a medicament in a pharmaceutical formulation that may be administered orally or transdermally, for achieving a reduction in and/or prevention of abuse of psychostimulants and/or opioids in an individual.

### Second Active Ingredient

The compounds of the present invention may be used in combination with other medicaments/compounds. The purpose hereof may be to increase the effect of the administered NPY Y5 antagonist or medicament comprising the NPY Y5 antagonist, to increase the efficacy of the treatment itself, or to alleviate or prevent symptoms associated with abuse/use of psychostimulants and/or opioids (such as withdrawal symptoms) or a co-dependency such as a dependency on tobacco and/or alcohol or other drugs.

In one aspect of the present invention NPY Y5 receptor antagonists are used in combination with each other to optimize the effect of administering a medicament comprising at least one NPY Y5 receptor antagonist. The medicament may be manufactured to comprise two or more Y5 receptor antagonists, such as three, four, five or more NPY Y5 receptor antagonists. Alternatively, the NPY Y5 receptor antagonists may be administered separately each comprised within its own pharmaceutical form, such as a pill, injectable liquid, inhalant, or other.

Other compounds to be used in combination with the NPY Y5 receptor antagonists of the present invention include, but are not limited to (trade names/synonyms in parenthesis): Naltrexone (Revia, Depade, Vivitriol); Acamprosate (Campral); Buprenorphine (Suboxone, Subutex); Cannabinoid antagonists such as Rimonabant (SR141716, Acomplia, Riobant, Slimona, Rimoslim, Zimulti); Varenicline (Chantix, Champix); Bupropion amfebutamone, Wellbutrin, Zyban); Methadone (Methadose, synthetic opiod); Dextropropoxyphene; Levomethadone; and Aripiprazole.

Any of the above second active ingredients may be used as sole second active ingredient together with a NPY Y5 antagonist or several second active ingredients may be used in combination together with a NPY Y5 antagonist. Alternatively two or more NPY Y5 antagonists may be used together with a single second active ingredient or in combination with several second active ingredients. Preferably, at least one NPY Y5 antagonist is used with buprenorphine or naltrexone or both.

### Dosage and Dosing Regimes

When the medicament comprising the NPY Y5 receptor antagonist is used, its dosage and the number of times of its administration vary depending on the sex, age, body weight and the conditions of the individual and the intended therapeutic effect. Furthermore, the dosage requirements will vary with the particular drug composition employed, the route of administration and as stated above, the particular individual being treated. Ideally, an individual treated by the present method will receive a pharmaceutically effective amount of the NPY Y5 antagonist in the maximum tolerated dose.

The intended therapeutic effect of administering a medicament comprising a NPY Y5 antagonist a herein described is achieving a reduction in and/or prevention of abuse of psychostimulants and/or opiods in an individual. If an individual has a repeat history of drug abuse such as abuse of any of the herein mentioned psychostimulants or opiods the therapy may be targeted towards a reduction or termination in the abuse of these drugs. By repeat history is meant a long-term and/or substantial abuse of drugs. In the situation where an individual never or only to a limited degree has been abusing or using drugs, the therapy will be aimed at preventing abuse of psychostimulants and/or opiods.

The medicament of the present invention may be administered several times a day, once a day, several times a week, such as twice or more a week, once a week, or several times a month, such as twice or more a month, or once a month. The medicament may be administered as a controlled release formulation. The choice of dosing regiment lies with a person skilled in the art.

The medicament of the present invention is given to reduce drug abuse and/or to prevent drug abuse. By reducing drug abuse it is understood, that the individual will lessen the intake of the abused drug(s) especially in regards to the amounts taken and/or reduce the frequency of intake. It is preferred that the medicament is administered to a person using/abusing drugs such as psychostimulants and/or opioids to reduce drug use/abuse. By preventing drug abuse it is understood that the individual will abstain from taking the drug of abuse.

In a preferred mode of administering the medicament of the present invention, the NPY Y5 antagonist is given prior to a situation in which psychostimulants and/or opiods are taken by the individual, i.e. the medicament may be given as a prophylactic. The medicament will thus be given to prevent drug abuse. Administered in this manner, the medicament of the present invention is comparable with disulfuram (Antabus or Antabuse), which is administered to prevent alcohol intake. For example, the medicament is used prior to analgesic treatment with opioids; preferably, the medicament is given to drug users and most preferably the medicament is given to rehabilitated drug users.

The dose administered should be an “effective amount” or an amount necessary to achieve an “effective level” in the individual patient. Since the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the patient that corresponds to a concentration of one or more compounds according to the invention.

Using oral and other administration routes, Y5 receptor antagonists will be effective for treatment of addiction to psychostimulants, opioids, and/or related drugs at doses of 0.01 to 1000 mg per kg body weight, such as doses between 0.1 to 500 mg/kg, 1 to 100 mg/kg, or such as 5 to 50 mg/kg body weight. Furthermore, the medicament may be administered in doses between 0.1 up to 300 mg per kg body weight, or in doses between 1 to 500 mg/kg, or 10 to 1000 mg/kg. When it is administered to an adult, it is desirable in general to orally administer in an amount of from 0.1 to 300 mg/kg per day by dividing the daily dose into 1 to several times per day, or to parenterally administer in an amount of from 0.1 to 300 mg/kg by dividing the daily dose into 1 to several times per day.

Preferably, the medicament comprising the NPY Y5 receptor antagonist will be administered in doses between 0.1 up to 300 mg per kg body weight. The medicament may be administered orally in a pharmaceutical form that allows/enables crossing of the blood brain barrier by the active ingredient.

Most preferably, the present invention provides means for the use of a NPY Y5 receptor antagonist for the preparation of a medicament for achieving a reduction in and/or prevention of abuse of psychostimulants and/or opioids in an individual, in which the medicament is orally administered at a dose between 0.1 up to 300 mg per kg body weight in a pharmaceutical form that allows crossing of the blood brain barrier. Also it is preferred that the medicament is given for the reduction in and/or prevention of abuse of psychostimulants and/or opioids in an individual in danger of relapsing into a drug abuse, i.e. an individual that otherwise is under rehabilitation or considered rehabilitated.

### Kit of Parts

Another embodiment of the present invention comprises a kit of parts, wherein the kit includes at least one NPY Y5 receptor antagonist according to any of the above, a means for administering said compound and the instruction(s) on how to do so. It is within the scope of the present invention to include multiple dosages of the same composition or several different compositions. In a preferred embodiment the kit of parts further comprises a second active ingredient. The second active ingredient may be administered simultaneously with the NPY Y5 antagonist or independently here from.

### DETAILED DESCRIPTION OF THE DRAWINGS

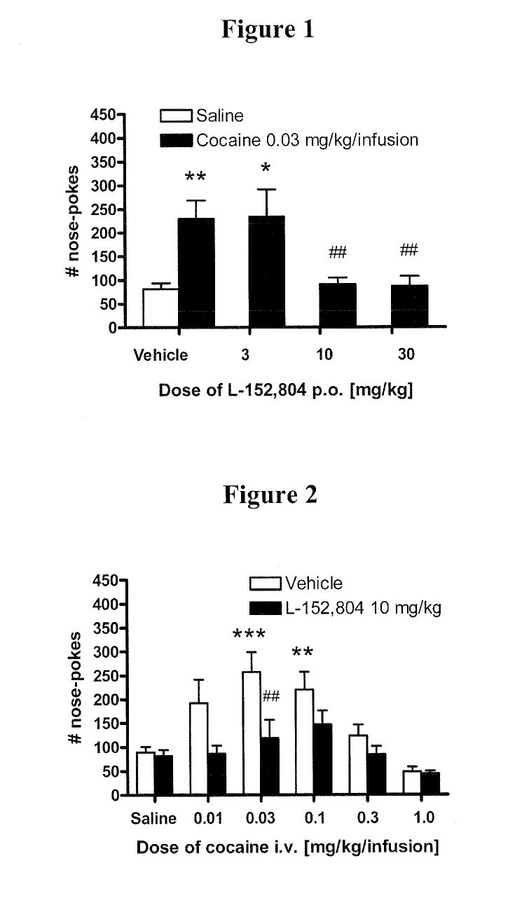

### FIG. 1 Dose-Dependent Reduction of Acute Cocaine Self-Administration.

L-152,804 pre-treatment p.o. (per oral) dose-dependently decreased nose-poking for cocaine at the optimal i.v. self-administration dose of cocaine (0.03 mg/kg/infusion) in mice, reaching significance at 10 and 30 mg/kg. **P<0.01, *P<0.05 vs. saline i.v. preceded by vehicle p.o., ##P<0.01 vs. cocaine i.v. preceded by vehicle p.o.; post-hoc LSD t-test following significant one-way ANOVA.

### FIG. 2 Effect of L-152,804 on the Self-Administration Dose-Response Curve.

Inverted U-shaped i.v. cocaine self-administration curves in mice pretreated with L-152,804 at 10 mg/kg or vehicle p.o. Cocaine's dose-effect curve was shifted down. There was no effect of L-152,804 per se, suggesting that its inhibitory action is due to an effect on cocaine self-administration rather than a non-specific effect on motor behaviour. ***P<0.001, **P<0.01 vs. corresponding saline i.v. group, post-hoc LSD t-test following significant one-way ANOVA after significant cocaine treatment effect in two-way ANOVA. ##P<0.01 vs. corresponding p.o. vehicle-pre-treated group, post-hoc test following significant L-152,804 treatment effect in two-way ANOVA.

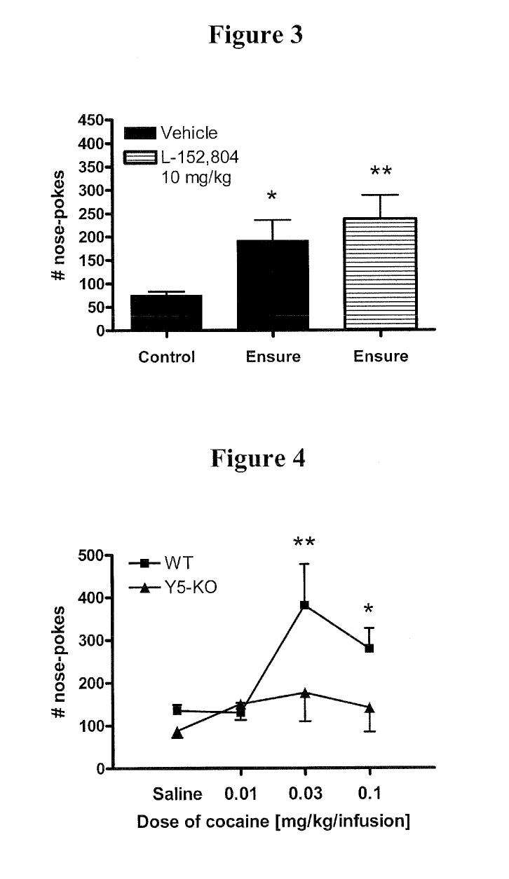

### FIG. 3 Effect of L-152,804 Pre-Treatment.

L-152,804 pre-treatment p.o. had no effect on food self-administration. **P<0.01, *P<0.05 vs. control, post-hoc LSD West following significant one-way ANOVA.

### FIG. 4 Self-Administration of Cocaine in WT and Y5-KO Mice.

WT mice self-administered cocaine as compared to saline while there was no significant cocaine self-administration in the Y5-KO mice. **P<0.01, *P<0.05 vs. corresponding saline i.v. group, post-hoc LSD t-test following significant one-way ANOVA after significant cocaine treatment effect in two-way ANOVA.

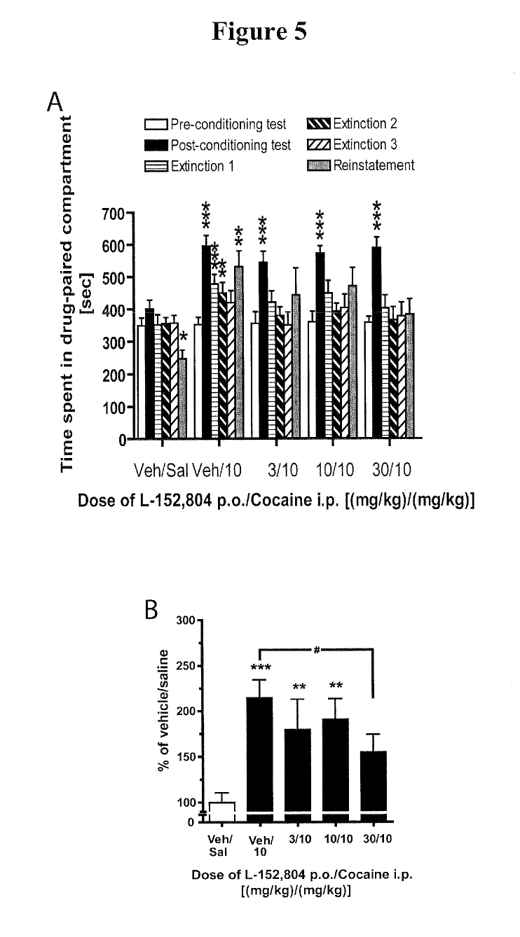

### FIG. 5 Effects of Y5 Antagonist on Cocaine Induced Conditioned Place Preference (CPP).

Effects of the Y5-antagonist, L-152,804, on cocaine-induced conditioned place preference. (A) L-152,804 did not attenuate the induction of place preference, but extinction was significantly faster at all tested doses and reinstatement of place preference was absent. ***P<0.001, **P<0.01, *P<0.05 vs. own pre-conditioning test, paired t-test following significant repeated measures one-way ANOVA. (B) Reinstatement presented as percent of the vehicle p.o./saline i.p. group. L-152,804 pre-treatment dose-dependently attenuated reinstatement of cocaine-induced place preference. ***P<0.001, **P<0.01 vs. vehicle p.o./saline i.p., #P<0.05 vs. vehicle p.o./cocaine i.p., post-hoc LSD t-test after significant one-way ANOVA.

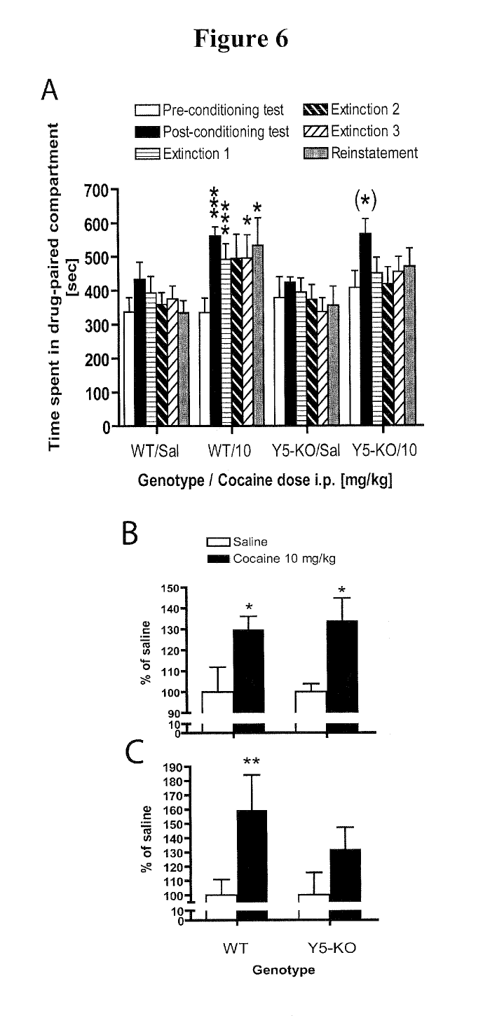

### FIG. 6 Cocaine-Induced Conditioned Place Preference Studied in Y5-KO and WT Mice

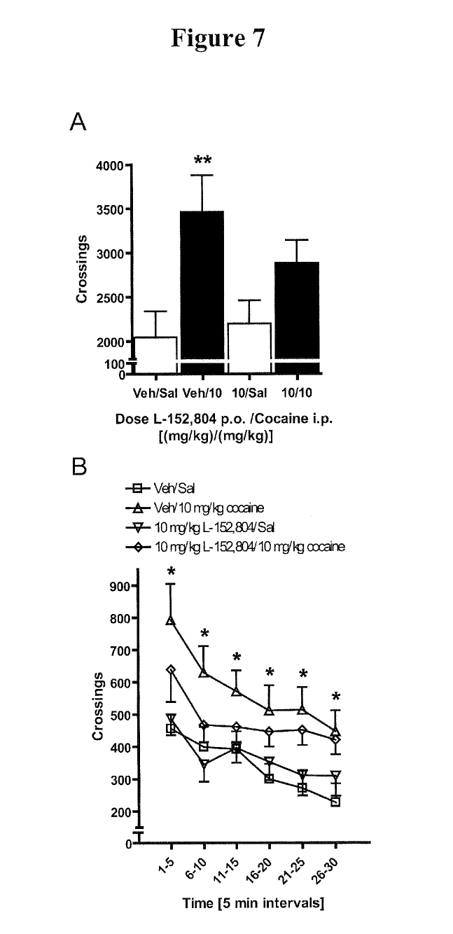

(A) Cocaine induced place preference in WT mice, but overall ANOVA was not significant in Y5-KO. ***P<0.001, **P<0.01, *P<0.05 vs. own pre-conditioning test, paired t-test following significant repeated measures one-way ANOVA. (B) The post-conditioning test presented as percent of its corresponding saline group. Cocaine conditioning was present in both Y5-KO and WT mice. *P<0.05 vs. corresponding saline, post-hoc LSD t-test after significant one-way ANOVA. (C) Reinstatement presented as percent of its corresponding saline group. Reinstatement was significant in WT mice but not in the Y5-KO mice. **P<0.01 vs. WT/saline, post-hoc LSD t-test after significant one-way ANOVA.
   FIG. 7 Cocaine Induced Significant Hyperactivity in Vehicle Pre-Treated Mice but not in Mice Pre-Treated with the Y5 Antagonist, L-152,804.
(A) Cocaine-induced hyperactivity presented as total counts for the 30 minutes observation period. **P<0.01 vs. vehicle/saline, post-hoc LSD test after significant ANOVA. (B) Cocaine-induced hyperactivity presented in 5 min intervals. Cocaine-induced hyperactivity appeared to be attenuated by L-152,804 throughout the full 30 minutes of testing. *P<0.05 vs. corresponding saline, independent t-test after significant two-way rANOVA and significant treatment effect, LSD post-hoc t-test.

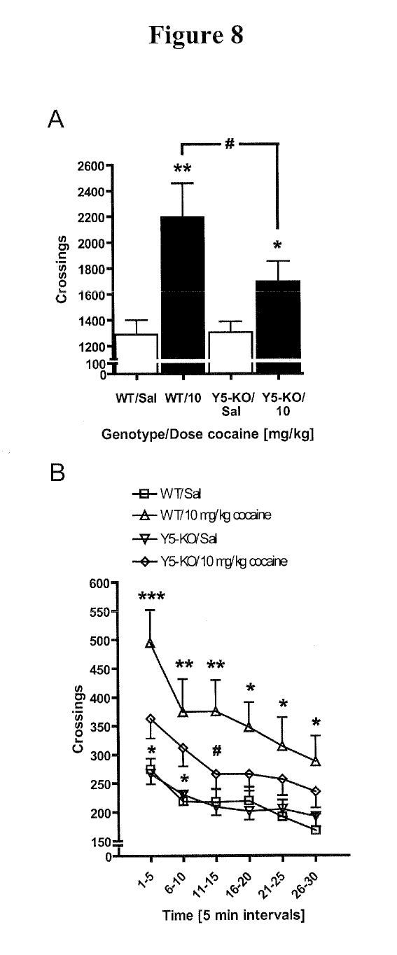

### FIG. 8 Cocaine-Induced Hyperactivity was Significantly Attenuated in Y5-KO as Compared to WT Mice.

(A) Cocaine-induced hyperactivity presented as total counts for the 30 minutes observation period. **P<0.01, *P<0.05 vs. corresponding saline group; ^{#}P<0.05 vs. Y5-KO/10 mg/kg cocaine, post-hoc LSD t-test after significant one-way ANOVA. (B) Cocaine-induced hyperactivity presented at 5-min intervals. Hyperactivity was induced at all 5-min intervals in WT mice while it was only present at the first two intervals in Y5-KO mice. ***P<0.001, **P<0.01, *P<0.05 vs. corresponding saline, ^{#}P<0.05 vs. Y5-KO/10 mg/kg cocaine, independent samples t-test after significant treat-ment effect in repeated measures two-way ANOVA LSD post-hoc t-test and subsequent overall LSD post-hoc t-test.

### EXAMPLES

Throughout this text the following abbreviations are used:

° C. or C: Degree centigrade
i.p. intraperitoneal
i.v. intravenous
p.o. per oral

The examples have been conducted using the following:

### Materials and Methods

### Experimental Animals

Male NMRI and C57Bl/6 mice (Taconic, DK) as well as male and female transgenic Y5-KO mice and littermate WT controls (Naveilhan et al, 2001; Fetissov et al, 2004; Woldbye et al, 2005) were used in the experiments. The transgenic mice were maintained on a mixed genetic background (Balb/c x129/SvEv, 75%/25%, respectively). The animals were group-housed under standard laboratory conditions in boxes of up to 10 mice per cage, on a 12 h light/dark cycle with free access to standard rodent chow and tap water. All experiments were performed between 0900 h and 1600 h. Animals were only used for one type of experiment listed below. The guidelines of the Animal Experimentation Inspectorate of Denmark were followed in all experiments.

### Compounds

The Y5 receptor antagonist, L-152,804 [5,5-Dimethyl-2-(2,3,4,9-tetrahydro-3,3-dimethyl-1oxo-1H-xanthen-9-yl)-1,3-cyclohexanedione] (Tocris Cookson Ltd., UK; #1382) was suspended in 0.5% methylcellulose (Acros Organics, BE; CAS no.: 9004-67-5) in distilled water by ultrasonification. Cocaine obtained from HS Pharmacy (Copenhagen, Denmark) was dissolved in 0.9% saline. The drugs were prepared immediately before use.

### Acute Self-Administration: Cocaine Reinforcement

The self-administration procedure was described previously (Sørensen et al, 2008a). Mice weighing 20-22 g were used. The self-administration apparatus consisted of transparent plastic boxes (8×8×8 cm) with a centred frontal nose-poke hole (12 mm diameter) 1 cm above the floor and a centred posterior vertical opening (width 5 mm) through which the tail extended. Dual photocells projected an infrared beam 1 mm in front of the nose-poke hole. Eight mice were tested at the same time, with interposed shields, preventing the mice from seeing each other during the experiment. Immediately before being placed in the test boxes, the mice were left for approximately 3 min 30-35 cm below a 150 W infrared light bulb to induce vasodilatation of their tails, thus facilitating the insertion of the cannula into their tail veins. The tail was fixed onto a stripe of double-sided adhesive tape (Tesafix® 50X19 4964, Lynge Papir & Kontor, Silkeborg, DK). The tape also fixed the tail at its base as well as the vein cannula at the cannula insertion point. A nose-poke interrupted the infrared beam, thus activating the photocell connected to an interface (SG 502, Med Associates Inc., East Fairfield, Vt., USA) and thereafter a syringe pump (PHM-100A, Med Associates Inc.) holding a 2 ml syringe (Omnifix® Solo, B. Braun, Copenhagen, DK) connected by a 75 cm PVC tubing (0.9 mm inner diameter, Original-Perfusor Leitung MR, B. Braun; Germany) to a 27 G infusion needle (0.4×40 mm, Sterican®, B. Braun). A back-check valve (Rüchschlagsventile, B. Braun) between needle and tubing prevented reflux of blood. A fixed ratio 1, FR-1, schedule was used, with no time-out between nose-poke and infusion, so that each nose-poke induced the i.v. infusion of 1.4 μl of drug. A computer and self-administration software (Ellegaard Systems, DK) were used to control infusions and to record nose-poking behaviour. After placing the mouse in the self-administration box for a 10 min habituation period during which nose-poking did not induce infusions, one priming infusion was given by the experimenter immediately before starting a 30-min session. Immediately after the session, the correct placement of the infusion needle was verified by manual infusion of the tested drug by an experimenter blind as to the number of nose-pokes produced, and the animals were quickly sacrificed. Mice were excluded from further analysis if they had not produced at least five nose-pokes during the self-administration session or correct placement of the infusion needle could not be verified.

Three self-administration experiments were performed. In the first, NMRI mice received p.o. administration of the Y5 antagonist L-152,804 at doses of 3 (n=18), 10 (n=18), or 30 (n=16) mg/kg or corresponding vehicle (nvehicle=18, ncocaine=23). Two hours later, the animals were subjected to the self-administration procedure described above with access to i.v. administration of vehicle or cocaine at 0.03 mg/kg/infusion, previously shown to be the optimal dose for self-administration (Sørensen et al, 2008). In the second experiment, NMRI mice received p.o. administration of 10 mg/kg Y5 antagonist or corresponding vehicle. Two hours later, the animals were subjected to the self-administration procedure described above with access to i.v. administration of cocaine doses of 0.01 (nvehicle=17, nL-152,804=17), 0.03 (nvehicle=19, nL-152,804=14), 0.1 (nvehicle=16, nL-152,804=17), 0.3 (nvehicle=14, nL-152,804=13), or 1.0 (nvehicle=12, nL-152,804=11) mg/kg/infusion or vehicle (nvehicle=21, nL-152,804=10). In the third experiment, female Y5-KO and WT mice were subjected to the self-administration procedure described above with access to i.v. administration of cocaine doses of 0.01 (nWT=10, nY5-KO=7), 0.03 (nWT=8, nY5-KO=5), or 0.1 (nWT=9, nY5-KO=8) mg/kg/infusion or vehicle (nWT=16, nY5-KO=10).

### Acute Self-Administration: Food Reinforcement

The self-administration apparatus used was described above. The end of the PVC tubing, mentioned above, was secured on the outside of the self-administration box at the top of the nose-poke hole, thereby enabling the mice to reach the end of the PVC tube to acquire the content used as reinforcer (ensure protein drink, Abbott Laboratories, USA). The animals fasted overnight, with water still available.

Male NMRI mice received p.o. administration of L-152,804 (10 mg/kg) or corresponding vehicle. Two hours later, the animals were subjected to the self-administration procedure described above with access to PVC tubing containing ensure (nvehicle=11, nL-152,804=13) or without any drink content (=control, n=16).

### Conditioned Place Preference

The CPP apparatus consisted of eight parallel set-ups, each consisting of two separate compartments measuring 19×19×19 cm. The walls extended upwards with clear Plexiglas plates to a total height of 43 cm, having the function of preventing the mice from jumping out, and at the same time allowing light to enter. The two compartments differed in visual as well as tactile cues. In one compartment, the walls and floor were painted white, and the floor was smooth, whereas in the other compartment, the walls were black, and the floor provided texture by insertion of a grey Lego plate. During habituation, post-conditioning test, extinction, and reinstatement, the mice were free to move between the two compartments through a square hole (3.5×3.5 cm). During conditioning, this opening was closed by insertion of a wooden plate. The movements of the animals were measured with the video-tracking program EthoVision (Noldus, Netherlands). All animals displayed preference for the black compartment during the pre-conditioning test and were, consequently, conditioned with cocaine to the white compartment.

The CPP procedure consisted of the following consecutive phases: Habituation (2 days), conditioning (8 days), post-conditioning test (1 day), extinction (3 days), reinstatement test (1 day). Habituation (days 1-2): Each day the mice were placed into the white compartment of the CPP boxes with the door open and allowed to explore the compartments for 20 minutes to become habituated to the CPP apparatus. On the second day (i.e., the pre-conditioning test), the time spent in each of the two compartments (black or white) was recorded. This determined the preferred compartment of each mouse. No injections were given during these sessions.

Conditioning (days 3-10): Before eight daily 30 minutes conditioning sessions the animals received i.p. injections of either drug or isotonic saline, and the door between the two compartments was closed. Drug groups received drug injections on drug-days and were placed 2-5 min. later in their non-preferred compartment. On non drug-days, these animals received vehicle injections and were subsequently placed in their preferred compartment. All control animals received injections with vehicle each day and were placed in alternating compartments (black/white) on alternating days. The presentation of drug was counterbalanced such that half of the mice were injected with drug on days 3, 5, 7, 9 and the other half on days 4, 6, 8, 10.

Post-conditioning test (day 11): The test was conducted on the day after the last conditioning session. Mice were allowed free access to both compartments for 20 minutes during which the amount of time spent in each compartment was recorded as described above for the pre-conditioning test. No injections were given during this session.

As mentioned above, all animals were tested in a drug-free state. This allows for conclusions to be drawn regarding the effects of cocaine on reward-related learning. At the same time the confounding effects of interfering cocaine actions on motor behaviour was avoided.

Extinction and reinstatement: During an extinction period of three days the animals were exposed to the apparatus as during the post-conditioning test for 20 minutes each day with open doors and without prior injection. Obtainment of partial extinction was defined as either (1) the absence of a significant increase in the time spent in the cocaine-paired compartment during extinction as compared to the pre-conditioning test or (2) the presence of a significant decrease in cocaine-paired compartment time during extinction as compared to the post-conditioning test. Full extinction was defined as the simultaneous presence of both 1 and 2 on the same extinction day. In order to induce reinstatement of drug-seeking behaviour the animals of the drug groups were injected with half of the drug dose used during conditioning as priming, and the animals of the control group received a vehicle injection. Subsequently, the time spent in the previously drug-paired (non-preferred) compartment was recorded for 20 min.

Two place preference experiments were carried out. In the first experiment, male C57Bl/6 mice (10 weeks) received p.o. administration of L-152,804 at doses of 3 (n=13), 10 (n=13), or 30 (n=13) mg/kg or corresponding vehicle (nsaline=19, ncocaine=19) two hours prior to i.p. conditioning with 10 mg/kg cocaine or saline. In the second experiment, male Y5 transgenic mice (10-18 weeks) were conditioned i.p. with 10 mg/kg cocaine (nWT=14, nY5-KO=8) or saline (nWT=14, nY5-KO=9).

### Cocaine-Induced Hyperactivity

The apparatus for measuring locomotor hyperactivity consisted of 16 activity Plexiglas boxes (37×21×17 cm). Eight dual photocells placed throughout the length of each box, detected movement of the animals. A plate was placed on top of the box in order to keep the mice from jumping out during the sessions. Sawdust was placed in a thin layer on the floor of the box. All activity boxes were connected to an interface (SG 502, Med. Associates Inc., East Fairfield, Vt., USA), measuring each beam-break. A computer and activity software (Ellegaard Systems, DK) were used to record the results from each activity box.

In all activity experiments an i.p. injection of either vehicle or drug was administered 3-7 minutes before testing. The animal was then placed in an activity box and the activity was measured for 30 minutes. Two cocaine-induced hyperactivity experiments were carried out. In the first, male NMRI mice (10-12 weeks) received p.o. administration of 10 mg/kg L-152,804 (nvehicle=13, ncocaine=13) or corresponding vehicle (nvehicle=12, ncocaine=10) two hours prior to i.v injection of 10 mg/kg cocaine or saline. In the second experiment, male and female Y5-KO mice or WT littermates (10-18 weeks) were administered 10 mg/kg cocaine (nWT=10, nY5-KO=29) or corresponding vehicle (nWT=14, nY5-KO=23) five minutes prior to testing.

### Statistics

Data were analyzed using SPSS (SPSS Inc, USA). In the self-administration experiments, two animals from the L-152,804 experiment were excluded. One from the 10 mg/kg L-152,804/0.03 mg/kg/infusion cocaine group and one from the 10 mg/kg L-152,804/1 mg/kg/infusion cocaine group as an extreme values defined as more than three inter-quartile ranges above or below the 25th or 75th percentile, respectively (SPSS Inc., 2004). A high variance was seen at effective cocaine doses (0.03, 0.1 mg/kg/infusion) as compared to lower variance in saline and non-self-administered cocaine doses. The higher variance at effective cocaine doses was probably due to the fact that some animals failed to learn the self-administration paradigm during the single-session. To counter this variance in-homogeneity, the number of nose-pokes was square root transformed before statistical analyses. In the first and third self-administration experiments, data were analyzed using one-way analysis of variance (ANOVA) followed by post-hoc LSD t-test. In the second and fourth experiments, two-way ANOVA was used. Subsequently, after significant treatment effect of cocaine, data were analyzed using one-way ANOVA followed by post-hoc LSD t-tests. After significant treatment (L-152,804) or genotype (Y5-KO) effect in the two-way ANOVA, we used post-hoc t-tests to determine the cocaine doses at which the treatment or geno-type effect occurred. In addition, for the second self-administration experiment, polynomial regression of second order was performed on logarithm transformed cocaine doses to further analyze the inverted U-shaped cocaine dose-response curve. The dose at which the nose-poking activity was at its maximum (vertex) was xmax=−b/(2·a). To assess whether the Y5 antagonist shifted the cocaine dose-response curve to the right or left, a likelihood ratio test was carried out of the hypothesis that xmax did not depend on whether Y5 antagonist pre-treatment was given (m/e, software package R, version 2.4; R Development Core Team 2006).

Conditioned place preference data were analysed using repeated measure one-way ANOVA between days within each group on all four groups of both experiments. This was followed by paired t-tests for pre-conditioning test vs. post-conditioning test, extinction 1, extinction 2, extinction 3, or reinstatement. In case of a significant post-conditioning test, an analysis of post-conditioning test vs. extinction 1, extinction 2, extinction 3, or reinstatement was made. Post-conditioning test and reinstatement presented as percent of its own saline group were analysed with a one-way ANOVA followed by post-hoc LSD t-tests. Total crossings were analysed with a one-way ANOVA followed by post-hoc LSD t-tests. Data presented as 5 minutes intervals was analysed using repeated measures ANOVA with treatment as between subjects factor followed by LSD post-hoc t-test. In case of a significant difference between treatment groups in the LSD post-hoc t-test, an independent t-test was applied testing differences between groups in each interval.

Data is presented as means±standard errors of the means. The level of significance is p<0.05.

### Example 1

### The Neuropeptide Y Y5 Receptor Antagonist L-152,804 Blocks Acute Self-Administration of Cocaine in Mice

### Purpose

In the present study, the ability of the NPY Y5 receptor antagonist L-152,804 to block acute self-administration of cocaine was tested in mice. The model is useful for screening for the acute reinforcing properties of cocaine.

### Results

Acute self-administration of cocaine: effects of the Y5 antagonist L-152,804. In the first experiment (FIG. 1), oral administration of L-152,804 significantly and dose-dependently decreased self-administration of the 0.03 mg/kg/infusion cocaine dose (one-way ANOVA, F(4,88)=4.96, P=0.001), reaching significance at the two highest doses of L-152,804 (10 and 30 mg/kg; P=0.004 and P=0.002, respectively).

In the second experiment (FIG. 2), two-way ANOVA revealed a significant effect of treatment with cocaine (F(5.168)=7.29. P<0.001) as well as L-152,804 (F(1.168)=11.31, P=0.001) without interaction (P=0.20). In vehicle p.o. pre-treated mice, polynomial regression of second order showed that cocaine was self-administered according to an inverted U-shaped curve (F(1.75)=9.98, P=0.002; y=−4.6x²12.6x+5.9), reaching maximum at 0.03 mg/kg/infusion, consistent with previous work (Fink-Jensen et al., 2003; Sørensen et al., 2008a). Post-hoc LSD t-test following significant one-way ANOVA (F(5.93)=6.61, P<0.001) indicated that cocaine doses of both 0.03 and 0.1 mg/kg/infusion were self-administered as compared to saline i.v. (P<0.001 and P=0.005, respectively). Polynomial regression showed that L-152,804-pretreated mice could also be fitted to a parabola, like vehicle-pretreated mice (F(1.68)=8.71, P=0.004; y=−3.4x²−7.8x+6.1). Likewise, one-way ANOVA for L-152,804-pretreated animals administered various doses of cocaine i.v. was significant (F(5.75)=2.47, P=0.040), but post-hoc LSD t-tests showed no significant doses vs. saline. In accordance with the above-mentioned treatment effect of L-152,804 in the two-way ANOVA, the 10 mg/kg L-152,804 dose significantly reduced the nose-poking frequency of i.v.-administered cocaine at the peak dose of cocaine (0.03 mg/kg/infusion; t(30)=2.94, P=0.006) and showed a similar trend at the other of the two cocaine doses self-administered significantly as compared to saline i.v. (0.01 mg/kg/infusion) (FIG. 2). However, L-152,804 p.o. in comparison with vehicle p.o. did not significantly decrease nose-poking when saline was present as the i.v.-reinforcer.

To further analyse if L-152,804 changed the potency or efficacy of cocaine, the vertices of the dose-response curves for cocaine with or without L-152,804 at 10 mg/kg were compared after fitting to parabolas. A likelihood ratio test showed that the vertex of the cocaine dose-response curve of L-152,804 treated mice was neither shifted significantly to the right nor to the left (χ²=1.31, df=1, P=0.25), indicating that L-152,804 did not change the potency of cocaine. However, a second likelihood ratio test showed that the vertex of the cocaine dose-response curve of L-152,804 treated mice was shifted significantly downward (χ²=12.42, df=1, P<0.001), indicating a change in the efficacy of cocaine.

In the third experiment (FIG. 3), post-hoc LSD t-test following significant one-way ANOVA (F(2,35)=4.34, P=0.021) confirmed that ensure protein drink was self-administered as compared to control (P=0.043) and that L-152,804 at 10 mg/kg did not alter this self-administration (P=0.008 vs. control; P=0.573 vs. vehicle pre-treated ensure), indicating that L-152-804 acts specifically on the cocaine effect in the acute self-administration paradigm and not on general reinforcement mechanisms.

### Conclusion to Example 1

The present study showed that oral administration of the NPY Y5 receptor antagonist L-152,804 caused a significant reduction in acute self-administration of cocaine in mice. This suggests that L-152,804 blocks the acute rewarding effects of cocaine and is consistent with the concept that Y5 antagonists could be effective for treating cocaine addiction in humans. A significant amount of L-152,804 has been observed in the rat brain 2 hrs after oral administration of L-152,804 (10 mg/kg) (Kanatani et al., 2000) and 80% Y5 receptor occupancy in mouse brain has also been observed 2 h after oral administration of L-152,804 (Ishihara et al., 2006). Thus it is likely that orally administered L-152,804 in the present study entered the brain and that the presently observed effects were due to blocking of Y5 receptors in the brain. Because high levels of NPY and also NPY Y5 receptors have been found in the nucleus accumbens (NAc), a central region in addiction, one likely mechanism by which L-152,804 decreased cocaine self-administration is by attenuating the reinforcing effects of cocaine in the NAc.

Administration of L-152,804 vs. methylcellulose vehicle showed a tendency toward a rightward shift of the cocaine dose-response curve. However, at no dose of cocaine did L-152,804 treated mice show higher rates of cocaine self-administration than p.o.-vehicle treated mice. Indeed, L-152,804 treated mice did not self-administer cocaine at a significantly higher rate that saline i.v. vehicle at any dose of cocaine. Thus maximal effect of cocaine appeared to be decreased by L-152,804. This is important because it suggests that in the case of future therapeutic use of Y5 antagonists for treatment of cocaine abuse, the patients are not likely to just increase their ingestion of cocaine by taking higher, potentially lethal doses. No per se effect of L-152,802 was observed, suggesting that reduced self-administration of cocaine is not caused by a reduction in locomotor activity that might lead to decreased nose-poking activity, but rather is a specific effect on cocaine self-administration.

In conclusion, the present study showed that the use of a selective NPY Y5 antagonist, L-152,804 significantly reduced cocaine self-administration in mice. This indicates that NPY Y5 receptors may play a role in mediating the reinforcing properties of cocaine. It further indicates that antagonists for NPY Y5 receptors should be effective in future treatment of cocaine abuse.

### Example 2

### Effects of the Neuropeptide Y Y5 Receptor Antagonist L-152,804 on Chronic Self-Administration of Cocaine in Mice

### Purpose

The above experiments showed that treatment with an Y5 receptor antagonist blocks the rewarding effects of cocaine in an acute self-administration model and antagonized relapse in the CPP model. To extend these findings further, the effects of the Y5 antagonist L-152,804 is tested in a mouse model of chronic cocaine self-administration. There are several advantages of this model. Most importantly, the model more specifically mimics the situation of a chronic state of dependence seen in human cocaine addicts. Effects of Y5 antagonists on induction of dependence, maintenance, and reinstatement/relapse can be studied separately. Moreover, the model allows for testing whether the effects of Y5 antagonists are specific for drug abuse or also affects natural reward (e.g., feeding).

Acute self-administration of cocaine: effects in Y5-KO mice. In Y5-KO mice, two-way ANOVA showed a significant effect of treatment with cocaine (F(3.65)=3.89, P=0.013) as well as genotype (F(1.65)=8.23, P=0.002) without interaction (P=0.153). In wild-type (WT) mice polynomial regression of second order showed that cocaine was self-administered according to an inverted U-shaped curve (F(1.24)=4.65, P=0.041; y=−20.1x²−55.3x−19.3), reaching maximum at 0.03 mg/kg/infusion (FIG. 4). Post-hoc LSD t-test following significant one-way ANOVA (F(3.39)=5.90, P=0.002) confirmed that the doses 0.03 and 0.1 mg/kg/infusion were self-administered as compared to saline i.v. (P=0.001 and P=0.024, respectively). In contrast, one-way ANOVA showed that Y5-KO mice did not self-administer cocaine significantly (P=0.530) and polynomial regression of second order did not indicate an inverted U-shaped curve form for cocaine self-administration in Y5-KO mice (F(1.17)=0.16. P=0.693; y=−4.4x²−14.3x+0.7).

### Example 3

### Neuropeptide Y Y5 Receptor Antagonist L-152,804 Reduces Relapse and Causes Faster Extinction in Cocaine-Induced Conditioned Place Preference (CPP) in Mice

### Purpose

The above experiments showed that pre-treatment with an Y5 receptor antagonist blocked the rewarding effects of cocaine in an acute self-administration model. To extend these findings, the effects of the Y5 antagonist L-152,804 was explored in another animal model for testing the rewarding effects of cocaine, the conditioned place preference (CPP) model. Furthermore, the CPP model allows for the testing of relapse to cocaine seeking behavior. Preventing relapse is a serious issue in treatment of drug addiction, and showing inhibitory effects on relapse behavior provides additional important evidence that Y5 antagonists are useful in treatment of psychostimulant addiction.

The basis for the CPP experiments is a learned association between an environment and the effect of the drug. In this model, drug and vehicle injections are paired with different environments (e.g. different wall color and/or floor material). This procedure is repeated for several days. During these conditioning trials the animal develops an association between the state produced by the drug (e.g. reward comparable to mood elevation and euphoria in humans) and the environment present during the drug state. When the animal is subsequently tested in an apparatus containing the drug-paired environment in one compartment and vehicle paired environment in another, the animal will voluntarily move toward the drug-paired compartment if the drug is rewarding. The model has been validated in mice by drugs widely abused by humans including cocaine.

Conditioned place preference: effects of L-152,804. The group receiving 10 mg/kg cocaine i.p. during conditioning preceded by vehicle p.o. displayed significant place preference as revealed by significantly longer time in the cocaine-paired compartment during the post-conditioning test as compared to the pre-conditioning test (paired t-test: t(18)=−6.29, P<0.001 after significant repeated measures ANOVA: F(5.85)=8.76, P<0.001; FIG. 5A). During extinction days 1 and 2, the same group displayed partial extinction, i.e., the animals spent significantly shorter time in the cocaine-paired compartment as compared to the post-conditioning test (extinction 1: t(18)=3.67, P=0.002; extinction 2: t(18)=3.56, P=0.002) while cocaine-paired compartment time remained significantly elevated as compared to the pre-conditioning test (t(18)=−4.36, P<0.001 and t(18)=−3.25, P=0.004, respectively). On the last extinction day, these mice obtained full extinction, as defined by a significantly shorter cocaine-paired compartment time as compared to the post-conditioning test (extinction 3: t(18)=3.78, P=0.001) and by the fact that there was no longer a significant increase in the time spent in the cocaine-paired compartments compared to the pre-conditioning test (FIG. 5A). The next day, the re-administration of cocaine at half the dose of that administered during conditioning (5 mg/kg) resulted in reinstatement of place preference as revealed by mice spending significantly longer time in the cocaine-paired compartment during the reinstatement test as compared to the pre-conditioning test t(17)=−3.27, P=0.005; FIG. 5A).

In the control group of mice receiving saline during conditioning preceded by vehicle p.o., paired t-test (t(18)=4.07, P=0.001) after significant repeated measures ANOVA (F(5.90)=6.63, P<0.001) revealed a significant decrease in the time spent in the non-preferred compartment during reinstatement as compared to the pre-conditioning test while there was no difference on other test days (FIG. 5A). This decrease probably reflects acute effects of stress after oral administration during reinstatement (on other test days no oral administration of vehicle was given).

Treatment with all three doses of L-152,804 prior to cocaine caused significant place preference similar to that observed without the Y5-antagonist (FIG. 5A). Thus the animals spent significantly longer time in drug-paired compartment during the post-conditioning tests as compared to pre-conditioning (3 mg/kg L-152,804: paired t-test t(10)=−5.76, P<0.001, repeated measures ANOVA F(5.50)=3.99, P=0.004; 10 mg/kg L-152,804: paired t-test t(12)=−5.07, P<0.001, repeated measures ANOVA F(5.60)=5.26, P<0.001; 30 mg/kg L-152,804: paired t-test t(11)=−6.52, P<0.001, repeated measures ANOVA F(5.55)=11.25, P<0.001). In contrast to the cocaine group without antagonist, L-152,804 treatment at all three doses resulted in full extinction already on the first extinction day and remained on extinction 2-3. Thus significant decreases in the time spent in the cocaine-paired compartment were found as compared to the post-conditioning test (3 mg/kg L-152,804, extinction 1: t(10)=4.59, P=0.001; extinction 2: t(10)=5.82, P<0.001; extinction 3: t(10)=4.56, P=0.001; 10 mg/kg L-152,804, extinction 1: t(12)=3.17, P=0.008; extinction 2: t(12)=5.87, P<0.001; extinction 3: t(12)=3.71, P=0.003; 30 mg/kg L-152,804, extinction 1: t(11)=−6.52, P<0.001, extinction 2: t(11)=6.34, P<0.001; extinction 3: t(11)=4.56, P=0.001) and time spent in the cocaine-paired compartment was not significantly different during extinction days 1-3 as compared to the pre-conditioning test (FIG. 5A).

L-152,804 at all three doses prevented significant reinstatement of cocaine-induced place preference (FIG. 5A). In addition, since as mentioned above acute oral administration of vehicle followed by saline i.p. caused a significant decrease during the reinstatement test as compared to preconditioning (FIG. 5A), we also analyzed the effect of L-152,804 during reinstatement as percent of the vehicle p.o./saline i.p. group (FIG. 5B). One-way ANOVA (F(4.68)=5.58; P=0.001) followed by LSD post-hoc t-tests showed that only the highest dose of L-152,804 (30 mg/kg) blocked reinstatement as revealed by significantly lower reinstatement as compared to cocaine without Y5 antagonist and by the absence of significant reinstatement in comparison with the vehicle p.o./saline i.p. group (FIG. 5B).

### Conclusion to Example 3

The present study showed that treatment with the Y5 antagonist significantly reduced relapse to cocaine-induced CPP and was associated with faster extinction of the CPP response. Importantly, the fact that cocaine relapse was antagonized in the CPP model indicates that treatment with Y5 antagonists could prove particularly useful in preventing relapse to cocaine seeking behavior in human addicts. Preventing relapse is considered one of the key problems in treating psychostimulant and opioid addiction. Faster extinction associated with L-152,804 treatment in the CPP model furthermore suggested that Y5 antagonists may be useful for stopping ongoing abuse of psychostimulants.

### Example 4

Conditioned place preference: effects in Y5-KO mice. As expected, WT mice conditioned with 10 mg/kg cocaine i.p. displayed place preference during the post-conditioning test as compared to the pre-conditioning test analyzed using paired t-test t(14)=−4.85, P<0.001) following significant one-way ANOVA (F(5,65)=4.08, P=0.003; FIG. 6A). Unlike in the Y5-antagonist conditioned place preference experiment above, the WT mouse strain only developed partial extinction during the extinction period. Thus a significant decrease in the time spent in drug-paired compartment during extinction 1 compared to the post-conditioning test (413)=2.28, P=0.040), and during extinction 2 there was no significant place preference as compared to the preconditioning test. At both extinction 1 and 3, the WT mice spent significantly longer time in the cocaine-paired compartment as compared the pre-conditioning test (extinction 1: t(13)=−3.74, P=0.002; extinction 3: t(14)=−2.16, P=0.049) (FIG. 5A).

Y5-KO mice treated with 10 mg/kg cocaine during conditioning did not reach significance in repeated measures one-way ANOVA (F(5.35)=2.17, P=0.079), suggesting that place preference was not induced in the Y5-KO mice. However, the post-conditioning vs. the pre-conditioning test was significant in a paired t-test (t(7)=−3.09, P=0.018), indicating that this would be a spurious conclusion. We also analysed the post-conditioning test as percent of the corresponding saline group in each genotype, showing a similar conditioning response in Y5-KO and WT mice (P=0.029 and P=0.010, LSD post-hoc t-tests after significant one-way ANOVA F(3,41)=3.36; P=0.028; FIG. 6B). As observed in mice treated with the Y5 antagonist, no significant place preference remained on any of the three extinction days (vs. pre-conditioning test), and full extinction was observed already on the first extinction day (post-conditioning test vs. extinction 1, t(13)=2.28, P=0.040, FIG. 6A).

Further consistent with L-152,804 results, reinstatement was not present in Y5-KO mice analyzed as percent of corresponding saline group while WT mice showed significant reinstatement (P=0.288 and P=0.006, respectively; LSD post-hoc t-tests following significant one-way ANOVA, F(3.41)=3.36, P=0.028; FIG. 6C). Likewise, paired t-test in Y5-KO during reinstatement vs. its own pre-conditioning was not significant (t(7)=−1.26, P=0.248).

Neither WT nor Y5-KO mice treated with saline during conditioning were significant in repeated measures one-way ANOVA (F(5.55)=1.03, P=0.407 and F(5,40)=1.11, P=0.370, respectively; FIG. 6A).

### Example 5

Cocaine-Induced Hyperactivity: effects of L-152,804. To further study the role of the Y5 receptor in cocaine-induced responses, we tested the effect of L-152,804 on cocaine-induced hyperactivity in an activity test. As expected, i.p.-administered cocaine acutely induced hyperactivity in vehicle p.o. pre-treated mice as compared to saline i.p. during the total 30 min observation period (P=0.003, LSD post-hoc t-tests following significant one-way ANOVA F(3.44)=4.21; P=0.011; FIG. 7A). Likewise significant increases in beam crossings was observed during all 5-min intervals using t-tests after significant overall LSD post-hoc t-test (Veh/Sal vs. Veh/10 mg/kg L-152,804; P=0.003) following significant treatment effect in repeated measures two-way ANOVA (F(5,220)=18.08, P<0.001, interaction P=0.566) (FIG. 7B). In contrast, oral pre-treatment with 10 mg/kg L-152,804 prevented a significant cocaine-induced increase in locomotor activity during the total observation period (P=0.151, LSD post-hoc t-test; FIG. 7A) and during the individual 5-min intervals (FIG. 7B). No effect was found of the Y5-antagonist as compared to vehicle in saline i.p.-treated animals.

### Example 6

Cocaine-induced hyperactivity: effects in Y5-KO mice. Cocaine-induced hyperactivity in WT as well as in Y5-KO mice as compared to corresponding saline groups during the min observation period (P=0.002 and P=0.038, respectively, LSD post-hoc t-test after significant one-way ANOVA, F(3.72)=5.33; P=0.002) (FIG. 8A). However, the hyperactivity was significantly lower in Y5-KO as compared to WT mice (P=0.046, LSD post-hoc t-test; FIG. 8A). Analysed during 5-min intervals, cocaine induced hyperactivity throughout all 5-min intervals in WT mice whereas Y5-KO mice only showed an increase during the first two 5-min intervals when compared to its corresponding saline groups using t-tests after significant overall LSD post-hoc t-tests (WT/Sal vs. WT/10: P=0.002; KO/Sal vs. KO/10: P=0.038) following significant two-way repeated measures ANOVA (F(5.360)=25.62, P<0.001, interaction P=0.180) (FIG. 8B). Overall LSD post-hoc t-test also revealed a significant difference between WT and Y5-KO mice receiving cocaine (P=0.046), however, this only reached significance in a t-test at the 11-15 min interval (FIG. 8B).

### Conclusion to Examples 1 to 6

Here we investigated the role of the Y5 receptor in three different animal models of cocaine addiction-related behaviour: acute self-administration, conditioned place preference, and hyperactivity. We show that the Y5 receptor antagonist L-152,804 attenuates effects of cocaine in all three models, and that Y5-KO mice display a phenotype consistent with the inhibitory effects of L-152,804. These data demonstrate that Y5 antagonists are the therapeutics of choice in cocaine addiction and that Y5 receptors play a role in mediating reinforcement of cocaine.

10 mg/kg of L-152,804 tested against a dose-response curve of cocaine showed that the curve was shifted significantly down but not sideward. This indicates that L-152,804 predominantly reduced the efficacy and not the potency of cocaine. From a therapeutic perspective this is important because if, for instance, L-152,804 had caused reduced potency, this might only have resulted in addicted individuals administering higher doses of cocaine. A general decrease in the efficacy of cocaine, independent of dose, to induce self-administration is much more promising.

L-152,804 attenuated self-administration of cocaine without affecting the self-administration of saline, showing that the effect of the Y5 antagonist was not to decrease basal responding. Moreover, L-152,804 pre-treated mice self-administered food in the same manner as vehicle pre-treated mice, demonstrating that inhibitory effects of L-152,804 on cocaine self-administration were specific for cocaine and not a general effect on natural reinforcement, see example 1.

To further investigate the role of Y5 in ASA of cocaine we tested Y5-KO and WT mice. WT mice self-administered cocaine significantly, reaching a significant dose at 0.03 mg/kg/infusion, the same peak dose as the vehicle pre-treated NMRI mice in the L-152,804 experiment and as previously found (Fink-Jensen et al., 2003; Sørensen et al., 2008a). In contrast, Y5-KO did not self-administer cocaine significantly. This result support the findings of the L-152,804 experiments, further implicating the Y5 receptor in the regulation of the self-administration of cocaine, see example 2.

A significant amount of L-152,804 has been observed in the rat brain two hours after oral administration of L-152,804 (10 mg/kg) (Kanatani et al., 2000), and close to 80% Y5 receptor occupancy in the mouse brain has also been observed between one and four hours after oral administration of L-152,804 (Ishihara et al., 2006). Thus it is likely that p.o. administered L-152,804 in the present study entered the brain and that the presently observed effects were due to blocking of Y5 in the brain.

In cocaine-induced conditioned place preference, oral pre-treatment with L-152,804 did not affect conditioning at any dose tested, as seen when comparing the pre-conditioning test data of the vehicle/cocaine group with the groups receiving L-152,804. Thus all cocaine treated groups conditioned to the cocaine-paired compartment. The same was observed in Y5-KO and WT mice that both displayed similar levels of cocaine-induced place preference. In contrast, all tested L-152,804 doses (3, 10, and 30 mg/kg) resulted in faster extinction than the vehicle group. In accordance with this finding, Y5-KO mice displayed full extinction already on the first extinction day while WT mice only developed partial extinction during the extinction period. Reinstatement in conditioned place preference was initiated via a priming dose of cocaine (half the dose used during conditioning), and L-152,804 was found to dose-dependently decrease reinstatement. Similarly, WT mice showed significant reinstatement while Y5-KO mice did not.

Why Y5 antagonism (L-152,804 and Y5-KO mice) did not prevent conditioning to cocaine as expected from the acute self-administration and hyperactivity experiments may be explained by the different models used, and it could be relevant that conditioned place preference is induced over a longer time period (8 days). Thus, although Y5 antagonism reduces the reinforcing efficacy of cocaine, it is still perceived as reinforcing, and the animal will learn to associate a reinforcing effect of cocaine with the previously non-preferred compartment. Nonetheless, preventing reinstatement is considered one of the key problems in treating psychostimulant addiction (Stewart, 2008). The fact that cocaine reinstatement was antagonized in the CPP model suggests that treatment with Y5 antagonists could prove particularly useful in preventing reinstatement to cocaine seeking behaviour in human addicts. Faster extinction associated with L-152,804 treatment in the CPP model furthermore shows that Y5 antagonists are useful for stopping ongoing abuse of psychostimulants.

Most drugs of abuse have the ability to induce an increased level of activity, and the ability of addictive drugs to induce hyperactivity has been suggested to reflect the reinforcing effect of drugs (Wise & Bozarth, 1987). Thus the fact that cocaine-induced hyperactivity was reduced by pre-treatment with L-152,804 and in Y5-KO lends further support to the concept that Y5 receptors play a role in neural circuits mediating addiction-related processes. The fact that neither L-152,804 treated mice nor Y5-KO mice receiving saline i.p. differed in locomotion activity from saline i.p. and WT mice, respectively, is consistent with a previous report showing no change in distance travelled in rats treated with L-152,804 (Schroeder et al., 2003). This contributes to the effects found in this paper: that the effects are mediated via Y5 receptors directly affecting cocaine-induced behaviour and not via non-specific motor activation.

The mechanism of the attenuating effects of Y5 antagonism on cocaine addiction-related behaviour remains to be determined. One possible explanation could be that Y5 antagonism reduces extracellular dopamine levels in the nucleus accumbens. Thus direct reinforcing effects of NPY have been described with NPY causing conditioned place preference via intra-accumbal injection (Josselyn and Beninger, 1993). This effect was blocked by the dopamine receptor antagonist cis-flupenthixol, indicating that the effect of NPY was mediated through a dopaminergic mechanism. The idea of this is further supported by the increase of dopamine levels found in the striatum after i.c.v. injections of NPY (Heilig et al., 1990; Kerkerian-Le Goff et al., 1992). Additional support for this is found from a study where a putative Y5 agonist was centrally administered that showed an increase in dopamine release in the hypothalamus (Matos et al., 1996). In addition, we recently showed that NPY infusion into the nucleus accumbens shell region, a central region mediating reinforcement of cocaine and other addictive drugs, causes a prominent increase in extracellular dopamine (Sørensen et al., submitted). However, whether this effect on dopamine in the nucleus accumbens is mediated via Y5 receptors remains to be studied. Consistent with this concept, interneurouns in the nucleus accumbens contain NPY that may modulate dopamine release (Massari et al., 1988) and Y5 receptor-like immunoreactive cells are present in the nucleus accumbens (Wolak et al., 2003) and Y5 mRNA is present in the ventral tegmental area, containing dopamine projecting neurons (Parker and Herzog, 1999; Durkin et al., 2000).

Whether effects of NPY on learning could be involved, is not clear. Thus NPY has been associated with modulation of learning and memory that could affect the induction of place preference, extinction, and reinstatement. For instance, central administration of NPY and gene transfer of NPY attenuates long-term potentiation (Whittaker et al., 1999; Sørensen et al., 2008b; Sørensen et al., 2008c). I.c.v. injected NPY enhanced memory retention in T-maze footshock avoidance and step-down passive avoidance training (Flood et al., 1987, 1989) and decreased accuracy in conditional discrimination procedure (Cleary et al., 1994) and NPY overexpressing mice had impaired spatial learning in the Morris water maze (Thorsell et al., 2000). So far, the effect of NPY on learning and memory has been linked to the Y2 receptor (Redrobe et al., 2004) and no studies have explored a potential role of Y5 receptors in learning and memory. However, since Y5 antagonism (L-152,804 and Y5-KO mice) did not block the acquisition of cocaine-induction conditioned place preference, it seems less likely that modulatory effects on learning and memory play an important role in the inhibitory effects of Y5 antagonism on cocaine addiction-related behaviour.

In conclusion, the present study shows that Y5 antagonism attenuates addiction-related effects of cocaine. Y5 receptors are a new target for treatment of cocaine addiction and Y5 receptors play an important role in cocaine-induced behaviour. This testifies that the Y5 receptor is a target for the treatment of addiction to psychostimulants.

### Example 7

### Effects of the Neuropeptide Y Y5 Receptor Antagonist L-152,804 on Extracellular Dopamine Levels in the Nucleus Accumbens (NAc) after Cocaine or Morphine Administration in Mice

### Purpose

Administration of drugs of abuse, including cocaine, is associated with increased levels of the neurotransmitter dopamine in the key addiction brain region, the nucleus accumbens (NAc). To determine the potential brain mechanism by which Y5 antagonists could block addiction, it shall be explored whether oral administration of the Y5 receptor antagonist L-152,804 causes reduced levels of dopamine in the NAc following administration of cocaine in mice. This will further demonstrate that treatment with Y5 antagonists may interfere importantly with drug reward mechanisms in the brain.

### Materials & Methods

Male NMRI mice are stereotaxically implanted with guide cannulas for microdialysis probes (CMA/7) under sodium pentobarbital anesthesia (50 mg/kg) in the NAc (anterior +1.2 mm, lateral +1.0 mm, ventral −5.0 mm from bregma) according to the atlas of Franklin and Paxinos (1997). Dialysis probe placements are verified histologically at the end of each experiment, and experimental data are excluded if the membrane portions of the dialysis probes are outside the NAc.

At 7 days after implantation, microdialysis probes in freely moving mice are perfused with Ringer's solution (147 mM Na+, 4 mM K+, 1.26 mM Ca2+, 1 mM Mg2+, and 152.5 mM Cl⁻, pH 6.5) at 1 μl/min for 180 min. Dopamine baseline concentrations are obtained from average concentrations of three consecutive 20 min, 20 μl samples. These and subsequent 20 min, 20 μl dialysate fractions are analyzed using an AS-10 autoinjector (Eicom), high-performance liquid chromatography (HPLC), with a PPS-ODS reverse-phase column (Eicom) and an ECD-100 graphite electrode detector (Eicom). The mobile phase consists of 0.1 M phosphate buffer (pH 5.5) containing sodium decanesulfonate (500 mg/l), EDTA (50 mg/l), and 1% methanol. All procedures are approved by the Danish National Animal Experiments Inspectorate.

### Design for L-152,804 Experiments

The effects of L-152,804 on baseline and cocaine-stimulated extracellular dopamine levels in the NAc would be tested. After baseline dopamine concentration determination the animals will receive an oral injection of L-152,804 (1-30 mg/kg) or vehicle (i.e., 0.5% methylcellulose in distilled water). Two hours later the mice receive an i.p. injection of cocaine (5-10 mg/kg), morphine (10 mg/kg), or isotonic saline and the effect on extracellular dopamine levels in the NAc is measured.

### Results

Treatment with L-152,804 will reduce the ability of cocaine and morphine to cause increases in extracellular dopamine levels in the NAc. This study will offer a working mechanism for the inhibitory effect of Y5 antagonists on drug reward.

### Example 8

### Effects of the Neuropeptide Y Y5 Receptor Antagonist L-152,804 on Morphine-Induced Conditioned Place Preference in Mice

### Purpose

The effects of the Y5 antagonist L-152,804 to block the rewarding effects of morphine is tested in the (CPP) model described in planned experiment 1 above. This study will extend above-mentioned planned experiments by providing evidence that Y5 antagonists are useful also in treatment of dependence and addiction to opiates, another important class of drugs of abuse.

### Design for L-152,804 Experiments

The ability of L-152,804 to block the rewarding effects of morphine in the CPP model is tested in the following way. The CPP procedure is performed as described in example 3, but, on the conditioning days and relapse testing, all animals receive an oral injection of L-152,804 (3, 10, or 30 mg/kg) or vehicle (i.e., 0.5% methylcellulose in distilled water) at 2 hours before being injected with morphine (5-10 mg/kg) or isotonic saline i.p.

### Drugs

Morphine hydrochloride is acquired from the Danish Hospital Pharmacies (SAD).

### Results

Treatment with L-152,804 will reduce the rewarding effect of morphine resulting in reduced morphine-induced CPP both during testing and relapse.

1. A method for achieving a reduction in and/or prevention of abuse of psychostimulants and/or opioids in an individual in need thereof.

2. The use according to claim 1, wherein the medicament is administered for achieving a reduction in and/or prevention of abuse of psychostimulants and/or opioids in a individual that has a prior abuse history (prevention of relapse).

3. The use according to claim 1, wherein the NPY Y5 receptor antagonist is selected from the group of: L-152,804; MK-0557; the 2-methanesulfonamidephenyl)piperazine class of Y5 antagonists; N-[5-(4-chlorophenyl)-1H-pyrazol-3-yl]-2-indanecarboxamide; FMS586; 3-(5-benzoyl-thiazol-2-ylamino)-benzonitrile; CGP71683A; 5,6-dihydro-4H-3-thia-1-aza-benzo[e]azulen- and 4,5-dihydro-6-oxa-3-thia-1-aza-benzo[e]azulen derivatives; Triazine derivates; N-(Sulfon-amido)alkyl[tetrahydro-1H-benzo[e]indol-2-yl]amines; Arylsulfonamidomethyl-cyclohexyl derivatives; 2-[4-(8-methyl-2-oxo-4H-benzo[d][1,3]oxazin-1-yl)piperidin-1-yl]-N-(9-oxo-9H-fluoren-3-yl)acetamide 5p; 5,5-diphenyl-imidazolones; Cyclohexylureido NPY Y5 receptor antagonists; S 25585; 3-amido-9-ethylcarbazoles; JCF109; and GW438014 A.

4. The use according to claim 1, wherein at least one NPY Y5 receptor antagonist is L-152,804 or MK-0557 or any salt or derivative of these.

5. The use according to claim 1, wherein the medicament is administered to prevent use of psychostimulants and/or opioids.

6. The use according to claim 1, wherein the psychostimulants and/or opioids abused are selected from the group of: cocaine, D-amphetamine, methamphetamine, 3,4-methylene-dioxy-methamphetamine, heroin, morphine, methadone, fentanyl, sufentanil, remifentanil buprenorphine, codeine, ketobemidone, hydromorphone, nicomorphine, oxycodone, pethidine, tramadol, dextropropoxyphene, pentazocine, cyclazocine, and etorphine.

7. The use according to claim 1, wherein the psychostimulants and/or opioids abused are cocaine, heroin, and/or amphetamine or derivatives hereof.

8. The use according to claim 1, wherein the medicament is formulated for oral administration.

9. The use according to claim 1, wherein the individual is a human being.

10. The use according to claim 1, wherein the medicament further comprises a second active ingredient.

11. The use according to claim 1, wherein the medicament further comprises a second active ingredient is selected from the group of: Naltrexone, Buprenorphine, Cannabinoid antagonists, Varenicline, Bupropion amfebutamone, Wellbutrin, Zyban, Methadone, Dextropropoxyphene, Levomethadone, and Aripiprazole.

12. The use according to claim 1, wherein the medicament is comprised within a kit-of-parts.
